(19) United States
(12) Patent Application Publication
(10) Pub. No.: US 2024/0148542 A1
(43) Pub. Date: May 9, 2024

(54) Morning Erections Urine Collection Device

(71) Applicant: Claudiney Santos Borges, Spencer, MA (US)

(72) Inventor: Claudiney Santos Borges, Spencer, MA (US)

(21) Appl. No.: 17/981,573

(22) Filed: Nov. 7, 2022

(51) Int. Cl. A61F 5/453; A61F 5/44

(52) U.S. Cl. CPC A61F 5/453; A61F 5/4404

(57) ABSTRACT

A urinal aid in form of a funnel-shaped assembly with handles allows males to accurately aim the urine where the assembly can be placed by a user between the legs and directed into the height pre-adjusted to urinate freely into the proposed assembly and allows to keep the clothes and toilet rim clean after use. This eliminates wetting oneself and messing the floor around the toilet. Furthermore, the assembly can be washed and reused conveniently.

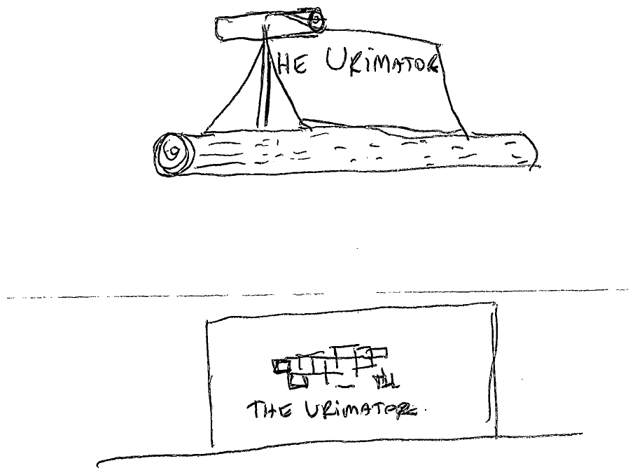

A portion of the disclosure of this parent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

This disclosure relates to systems, methods, and apparatuses for urinary aids, and particularly to a portable, re-useable urinal designed for males for directing and collecting urine, especially in case of morning erections.

### Description of the Related Art

Spontaneous awakening from sleep is significantly associated with the erection cycle and the association had already been documented in the early 1940s. Sleep is different for each individual, but most men have four to five full erections during REM sleep, with each erection lasting about 25-35 minutes. The erections are a normal part of REM sleep for males of all ages, even in infants and children. The relationship between REM sleep and erections is the reason that men are likely to awaken directly from REM sleep, or soon after a long morning REM period has ended. Thus, even though men do not always wake up at the end of each episode of REM sleep, they are very likely to awaken in the morning with a REM sleep-related erection. Morning erections are scientifically defined as involuntary sleep-related erections (SREs) or nocturnal penile tumescence (NPT) and are a healthy and normal physiological response that most men experience.

Erections are considered a normal part of sleep physiology in men. Evaluation of NPT is one of the earlier tests devised to study erectile dysfunction (ED). After demonstrating that NPT was a general phenomenon in healthy males 3 to 79 years old, Karacan, in the early 1970s, suggested that NPT could be used to evaluate ED. The mechanism of NPT is presumed to rely on neurovascular response mechanisms similar to those seen in erotically induced erections. Thus, men who are documented to have normal NPT are presumed to have a normal capacity for spontaneous, erotically induced erections. As a matter of fact, the primary function of NPT tests is to distinguish psychogenic causes of ED from organic causes. Men who do not have erections because of psychological problems can still have erections during deep sleep. If a lack of morning erections is accompanied by a lack of overall erections, a physical cause is suggested rather than a psychological cause. Most men from their teenage years to their older age will experience erection in the morning which is almost impossible to urinate without the possibility of wetting themselves.

By looking at prior art multiple types of advancements can be seen regarding urine collection assemblies. For instance, U.S. Pat. No. 5,893,176, is issued to Magiera. The patent is directed towards a female urine device to direct the discharge of urine while standing. The female urine device incorporates an inner sealing ring, an outer sealing ring and a funnel. The inner sealing ring is adapted to surround and sealingly engage the exterior of the labia minora. The outer sealing ring lies adjacent the inner sealing ring and is adapted to surround and sealingly engage the exterior of the labia majora. The funnel is located below the inner and outer sealing rings and it is adapted to direct the discharge of urine.

U.S. Pat. No. 5,091,998, is issued to Witzke. The patent is directed towards a funnel device to facilitate urination by women in an upright position includes a semi-rigid funnel rim contoured to surround the female genital region. A flexible funnel body depends from the rim and is sealed to the rim around the circumference. The funnel body has a continuous wall sloped inwardly and towards the front of the funnel body. The funnel body terminates in an orifice situated toward the front of the funnel body. A bendable, elongated disposal tube is sealably attached to the funnel orifice and is inclined downward and outward at an angle with regard to the funnel body. The tube is circumferentially corrugated to enable bending for storage and for directing urine flow.

U.S. Patent Application No. 20020193762, is issued to Suydam. The patent is directed towards a hand-held disposable portable urinal device particularly well suited for female use. The easy-to-use device has a leak and splash resistant soft upper seal attached to a retractable funnel, which easily folds back into a sealable bag. The funnel has a neck portion which allows the user to comfortably hold the urinal close to the ureter during use. The disposable urinal bag is odor- and vapor-sealable, and has a hydrophobic polymer containing inner bag for absorbing the urine. In addition, the disposable urinal has a deodorizer activated by the urine upon contact. The disposable urinal is easily transported in a folded state, and preferably comes three in a box. After use, the disposable urinal may be sealed and thrown away.

Another patent on Disposable funnel for urine samples bearing US patent 2,007,0006368A1 is issued to Lori Key. The patent implies a disposable urine funnel is a urinary aid device that is placed upon a vaginal opening when the user is seated in order to guide urine into a urine sample cup. The disposable urine funnel is a bowl-shaped container that may be integrally connected to a tube, or the tube may be omitted entirely. The container has an upper lip that defines a mouth of the container. The mouth of the container has a first end and a second end, the first end being narrower from one side of the mouth to the other than the second end. The tube is optionally attached to the container and extends angularly downward from the container and in the direction of the first end of the mouth of the funnel, terminating in a discharge opening from which urine passes.

Another patent on Flush toilet with a urine funnel bearing US patent 2,011,0004991A1 is issued to Yu-Chou Tai. The patent discusses a flush toilet with an additional urine funnel comprises a bowl, a urine funnel, a toilet seat and a delivery tube. A concave portion is provided at frontal side of the bowl sized for receiving said urine funnel. Said toilet seat is coupled to the bowl pivotally and covers the urine funnel. The delivery tube is connecting with the urine funnel and said urine funnel can be ascended to a predetermined height as the toilet seat is lifted.

A US patent 2,007,0169253A1 on Funnel for female urine direction is issued to Lisa Panzer. The patent discusses a funnel used by a woman to direct urine. A woman can use the funnel while urinating by urinating directly into the final, thereby directing the urine from the funnel into a toilet.

A U.S. Pat. No. 4,751,751A on Disposable urinating funnel for females is issued to Richard C. Reno. The patent implies a disposable urinating funnel for females comprises a thin, paper-like sheet which is folded in half to form two overlapping sections whose free edges are connected together and are tapered toward the fold line to form a funnel-like shape. The upper edges of the funnel-shape are curved to cover a female vaginal area, and the opposite, bottom edges form a spout-like portion. Score lines are formed midway between the fold line and connected side edges in each of the sections and extend upwardly from the bottom of the spout-forming portion toward the upper end of the funnel-like shape. Manually squeezing the fold line and connected side edges toward each other causes the otherwise flat funnel-like shape to open, by bowing the sections apart along the score lines. In addition, the tapered, connected-together edges are cut along a shallow curve which permits the funnel to be used with the fold line forwardly or rearwardly for selectively directing the flow of liquid through it.

There are multiple solutions that have been presented in prior art. However, these solutions are limited and restricted to their conventional architecture, installation system and have considerable shortcomings which adversely affect the convenience with which they can be used. The prior systems have certain limitations including the design of assemblies which make them not suitable for every type of user. Moreover, the ease-of-use ability of these assemblies is also questionable.

It has to be noted that the current invention proposes an assembly with an advancement where the design provides ease of use and enhanced options to males especially when they are facing more common episodes of NPT. The proposed design is funnel-shaped with a handle, is a portable, re-useable urinal aid designed for males. The mouth of the design has a beak to allow access, while the interior sides are broadly designed, allowing the container to be filled easily. Once comfortably in place, the person can then urinate freely without the worry of wetting/soiling.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a fill description. A full appreciation of the various aspects of the invention can be gained by taking the entire specification, claims, drawings, and abstract as a whole.

The primary desirable object of the present invention is to provide a novel and improved form of a urine collection device assisting males who are facing Nocturnal penile tumescence and aiding them in the urination process.

It is also the objective of the invention to provide a funnel-shaped, portable, re-useable urinal designed for males which is not only elegant in its design but is functional in nature.

Another object of the present invention is to provide a urinal assembly with a handle that can be carried easily by the person or caretaker.

Another object of this invention is to provide a new and improved assembly that provides a urinating man ease of use where he can adjust the assembly to his appropriate level to place his penis, urinate, and once relieved the user places the stopper in the mouth of the container where it can be left until a convenient time to dispose of the contents and wash.

Yet another object of the present invention is to provide a structure that is relatively inexpensive to produce so that ownership is available to a substantial majority of families.

It is also the objective of the invention to provide a smart assert b that provides ease of use and convenience to everyone.

It is a further aspect of the present invention to provide a new and improved assembly that is of durable and reliable construction and can sustain its effectiveness after multiple and consistent uses.

Thus, it is the objective to provide a new and improved urine collection device. Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

Fla 1 shows front view of claimed assembly as per preferred embodiments of the invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The device as per its preferred embodiments is a urinal aid designed to help Nocturnal penile tumescence patients/people urinate while avoiding wetting/soiling their garments and avoiding bacterial infection.

The proposed assembly as per its preferred embodiments is a stylish, portable, re-useable urinal for males, 1.2 L in volume. It is made of high-quality plastic and with an opening to receive urine from the user in a comfortable position. An extra-large silicone stopper with protruding sides is used in conjunction to create a sealable container. The device's primary function is to provide convenience to the user, especially to Nocturnal penile tumescence patients. Its secondary function is to alleviate some of the stigmas that comes with the action required.

The method of use of propped assembly is when user needs to urinate, he will remove the stopper and urinates into the container. Once relieved, the user seals the container with the stopper and prepares for later disposal of contents and cleaning.

The assembly as per its additional embodiments has a design that is very practical in nature. The proposed assembly has a short beak to allow access, while the interior sides allow the container to be filled easily.

The proposed assembly is made of a combination of materials including plastic, and it can be used by general masses and patients to assist them in keeping their garments dry/clean and still be able to urinate without concern and avoid bacterial contamination.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1: A urine aid assembly to facilitate directional urination and cleansing by males or by people facing morning erections, comprising:
A funnel-shaped assembly;
A short beak to allow access;
A broader base to hold the liquid; and, A handle to hold the assembly conveniently.

2: A device allowing males to accurately aim their urine into a proposed assembly where the user removes the winged stopper and places his penis in the mouth of the container and proceeds to urinate. Once relieved the user places the stopper in the mouth of the container where it can be left until a convenient time to dispose of the contents and wash.
